(19) United States
(12) Patent Application Publication
(10) Pub. No.: US 2008/0070986 A1
(43) Pub. Date: Mar. 20, 2008

(54) ALKYLETHER CITRATES FOR SELECTIVELY CLEANING THE SKIN

(76) Inventors: Annette Mehling, Wuppertal (DE); Bettina Kopp-Holtwiesche, Duesseldorf (DE); Hermann Hensen, Haan (DE)

Correspondence Address: COGNIS CORPORATION;PATENT DEPARTMENT, 300 BROOKSIDE AVENUE, AMBLER, PA, 19002, US

(21) Appl. No.: 11/850,368

(22) Filed: Sep. 5, 2007

(30) Foreign Application Priority Data
Sep. 19, 2006 (DE) 10 2006 044 618.6

(51) Int. Cl. A61K 31/19; A61K 31/20; A61P 17/00

(52) U.S. Cl. 514/558; 514/574

(57) ABSTRACT

The invention relates to the use of alkylether citrates in cosmetic and pharmaceutical preparations for protecting and maintaining the natural skin/mucous membrane microflora and the microbial skin/mucous membrane ecosystem without impairing the natural balancing processes which limit the number of pathogenic bacteria so that they do not become disease-inducing.

### RELATED APPLICATIONS

This application claims priority from DE 10 2006 044 618.6 filed Sep. 19, 2006; the entire contents of which are incorporated herein by reference.

### BACKGROUND OF THE INVENTION

This invention relates generally to cosmetic and pharmaceutical skin cleaning preparations and, more particularly, to preparations for selectively cleaning the surface of the skin using certain alkylether citrates.

### PRIOR ART

Since time immemorial, the object of cleaning the skin has to been to free the skin from adhering soils and thus inter alia to avoid the transmission of infectious bacteria and fungi. Over-frequent use of surfactants or antiseptic preparations such as, for example, shower baths, shower gels, rinses, shampoos, etc. leads to skin damage. The skin's own lipids are removed in exactly the same ways as the soils, which results in drying out of the skin. The skin becomes rough and dry. In addition, the natural skin flora are destroyed by frequent washing or undergo a change in their composition, i.e. the germs responsible for a healthy skin milieu are destroyed and/or removed. The skin microflora also play a major role in maintaining the acid protection jacket of the skin and mucous membrane. When the natural acid protection jacket of the skin is attacked or even destroyed, pathogenic germs are in danger of populating the skin or assuming the upper hand over the “natural” skin flora and leading to inflammation. Representatives of resident microorganisms can develop pathogenic properties through modified homeostasis. The natural skin flora consists of a heterogeneous spectrum of bacteria and yeasts/fungi dominated by gram-positive bacteria (for example Staphylococcus epidermidis, Propionibacterium acnes) or yeasts of the malassezia group. In addition, the composition of the skin microflora of each individual person is dependent upon body location and living conditions.

The problem addressed by the present invention was to provide surfactant formulations which would have little or no influence on the natural skin microflora because a healthy skin microflora plays an important part in the skin barrier function. At the same time, however, these formulations would be capable of effectively freeing the skin from adhering soils.

### BRIEF DESCRIPTION OF THE INVENTION

The present invention relates to the use of alkylether citrates as selective surfactants for cleaning the skin. In the context of the invention, selective surfactants are understood to be surfactants which attack the germs naturally occurring on the skin (bacteria and yeasts/fungi) to only a limited extent, if at all. These surfactants are used to protect and maintain the natural skin/mucous membrane microflora and the microbial skin/mucous membrane ecosystem without impairing the natural balancing processes which limit the number of pathogenic bacteria so that they do not become disease-inducing. They serve to protect the skin and the skin barrier function and to maintain the homeostasis. They are used to maintain the natural pH value (acid protection jacket of the skin) and to control the population of the skin by pathogenic germs and hence serve to maintain the natural skin ecosystem.

### DETAILED DESCRIPTION OF THE INVENTION

In the present context, the term “skin” encompasses both skin and mucous membrane. By skin microflora/skin flora/standing flora are meant the skin-populating microorganisms (bacteria, yeasts, fungi, viruses), the microbial ecosystem. They represent the microbial ecosystem of the skin which performs a barrier function. The term “skin cleaning” encompasses, for example, the following cosmetic and pharmaceutical preparations: body cleaning preparations, intimate care, shower baths, shower oils, shower gels, shampoos, conditioners, foam baths, baby shampoos, dry and wet cleaning wipes, creams and lotions, etc.

### Alkylether Citrates

Alkylether citrates are understood to be citric acid ester mixtures of ethoxylated alcohols corresponding to general formula (I):

R¹O(CH₂CH₂O)ₙH (I)

in which R¹ is an alkyl group and n stands for the degree of ethoxylation, R¹ being a linear C₆-C₂₂ alkyl group. However, this alkyl group may also be derived from a fatty alcohol mixture of natural or synthetic origin with different alkyl chain lengths. In a preferred embodiment, the alkyl group R¹ is derived from a fatty alcohol mixture containing 45 to 75% by weight C₁₂ alcohol, 15 to 35% by weight C₁₄ alcohol, 0 to 15% by weight C₁₆ alcohol and 0 to 20% by weight C₁₈ alcohol. n is a number of 5 to 9. The ratio by weight of monoester to diester in the citric acid ester mixtures is preferably in the range from 3:1 to 10:1.

### EXAMPLES

The scale of an antimicrobial effect of surfactants can be established in a biological test system in which the minimum inhibiting concentration (MIC) is comparatively determined under laboratory conditions. In a dilution series of the test substance, the MIC is just that concentration at which no further growth is possible. The milder a surfactant, the higher the MIC value.

The minimum inhibiting concentration of various surfactants was determined for three microorganisms occurring on the skin (Table 1). The surfactants are referred to by their names under the INCI nomenclature.

TABLE 1
Minimum inhibiting concentration of various surfactants
Test solution: 10% active substance, pH 6.5
Determined to DIN 58940 and DIN 58944

| | Sodium | | | |
|---|---|---|---|---|
| | Laureth | Cocamidopropyl | Lauryl | Laureth-7 |
| | Sulfate | betaine | Glucoside | Citrate |
| Propionibacterium | 0.05 | 0.05 | 0.05 | >0.5 |
| acnes | | | | |
| Staphylococcus | 0.5 | 0.1 | 0.5 | >0.5 |
| epidermidis | | | | |
| Malassezia furfur | >0.5 | >0.5 | >0.5 | >0.5 |

The alkylether citrate Laureth-7 is distinguished from the other surfactants by the fact that Propionibacterium acnes and Staphylococcus epidermidis are only inhibited at considerably higher concentrations.

Where alkylether citrates are used in combination with other surfactants, the selectivity of the alkylether citrates affects the germ killing potential of the formulation as a whole, i.e. the positive properties of the alkyl ether citrates on the maintenance of homeostasis or the growth properties of the microflora also have an effect on the composition as a whole. Instead of the reducing the skin's defence against pathogens, as conventional surfactants often do, it remains unchanged.

Table 2 shows minimum inhibiting concentrations of various mixtures of alkylether citrates with alkylether sulfates. It is clear that, despite the same active substance content, the formulations with an increasing content of alkylether citrates show increasing minimum inhibiting concentrations, i.e. have a reduced irritation potential and hence contribute towards maintaining or restoring a natural and healthy skin flora.

TABLE 2
Minimum inhibiting concentration of various surfactant combinations

| | AEC | AEC/AES | AEC/AES | AEC/AES | AES |
|---|---|---|---|---|---|
| | 100% | 75/25% | 50/50% | 25/75% | 100% |
| Propionibacterium | ≧1 | ≧1 | 0.5 | 0.1 | 0.05 |
| acnes | | | | | |
| Staphylococcus | ≧1 | ≧1 | ≧1 | 1.0 | 0.5 |
| epidermidis | | | | | |
| Malassezia | ≧1 | ≧1 | ≧1 | ≧1 | ≧1 |
| furfur | | | | | |

Test solution: 10% active substance
Determined to DIN 58940 and DIN 58944
AEC = alkylether citrate = Laureth-7 Citrate
AES = alkylether sulfate = Sodium Laureth Sulfate

We claim:

1. A skin-cleaning composition comprising: a mixture of esters of citric acid with ethoxylated fatty alcohols which when applied to skin protects and maintains a natural skin/mucous membrane microflora and the skin/mucous membrane ecosystem, without incurring the natural balancing processes which limits the number of pathogenic bacteria, so they do not become disease inducing.

2. The composition of claim 1, wherein, the ethoxylated alcohol is a composition of the formula:
R¹O(CH₂CH₂O)ₙH (I),
wherein, R¹ is an alkyl group containing from 6 to 22 carbon atoms, n is a number of from 5 to 9 and a ratio by weight of monoester to diester in the mixture of esters is in a range of 3:1 to 10:1.

3. The composition of claim 2 wherein R1 comprises residues of a mixture of fatty alcohols containing 45% to 75% by weight C₁₂ alcohol, 15% to 35% by weight C₁₄ alcohol, 0% to 16% by weight C₁₆ alcohol, and 0% to 20% by weight C₁₈ alcohol.

4. A method for cleaning skin which comprises: applying to the skin the composition of claim 1, whereby, the natural skin ecosystem is maintained.

5. A method for cleaning skin which comprises: applying to the skin the composition of claim 1, whereby, a natural pH value of an acid protection jacket of the skin is maintained and a population of pathogenic germs is controlled.

6. A method for cleaning skin which comprises: applying to the skin the composition of claim 1, whereby, the skin and skin barrier function is protected and homeostasis is maintained.

7. A method for cleaning skin which comprises: applying to the skin the composition of claim 2, whereby, the natural skin ecosystem is maintained.

8. A method for cleaning skin which comprises: applying to the skin the composition of claim 2, whereby, a natural pH value of an acid protection jacket of the skin is maintained and a population of pathogenic germs is controlled.

9. A method for cleaning skin which comprises: applying to the skin the composition of claim 2, whereby, the skin and skin barrier function is protected and homeostasis is maintained.
